(19) United States
(12) Patent Application Publication
(10) Pub. No.: US 2026/0315790 A1
(43) Pub. Date: Oct. 15, 2026

(54) Implantable Medical Device

(71) Applicant: BIOTRONIK SE & Co. KG, Berlin (DE)

(72) Inventors: Jens RUMP, Berlin (DE); Karsten SCHLODDER, Fuerstenwalde (DE)

(73) Assignee: BIOTRONIK SE & Co. KG, Berlin (DE)

(21) Appl. No.: 19/481,060

(22) PCT Filed: May 13, 2024
(86) PCT No.: PCT/EP2024/063017

§ 371 Date: Nov. 3, 2025

(30) Foreign Application Priority Data
May 16, 2023 (EP) 23173504.4

(51) Int. Cl. A61N 1/05; A61N 1/362; A61N 1/375; A61N 1/39

(52) U.S. Cl. CPC A61N 1/0573; A61N 1/0563; A61N 1/362; A61N 1/37518; A61N 1/3756; A61N 1/3968

(57) ABSTRACT

Electrode assembly for an implantable lead or leadless implant, wherein the assembly includes a tubular or cylindrical body extending in a longitudinal direction. The assembly further includes at least one spiral fixation member extending from the distal end of the body, wherein the spatial extension in radial direction of the at least one fixation member is configured such that, at zero-load, the respective fixation member protrudes in radial direction beyond the greatest outer circumference of the body, wherein an electrode member is provided at the distal end of the body and is configured to receive electrical signals from and/or to transmit electrical signals to a tissue at a pre-defined target position, wherein one of the at least one fixation member forms the electrode member or the electrode member is an element formed separately from the at least on fixation member and is located at a distal end of the body.

### CROSS-REFERENCE TO RELATED APPLICATIONS

This application is the United States National Phase under 35 U.S.C. § 371 of PCT International Patent Application No. PCT/EP2024/063017, filed on May 13, 2024, which claims the benefit of European Patent Application No. 23173504.4, filed on May 16, 2023, the disclosures of which are hereby incorporated by reference herein in their entireties.

### TECHNICAL FIELD

The invention relates to an implantable medical device, namely an implantable lead or a leadless implant, an electrode assembly for such implantable lead or leadless implantable device, a fixation method of such implantable lead or such leadless implant to a tissue at a pre-defined target location within a patient's body, and an implantation assembly for implanting such implantable lead or such leadless implant to a tissue at a pre-defined target location within a patient's body.

### BACKGROUND

Active or passive implantable medical devices, for example implantable intracardiac pacemakers or implantable leadless pacemakers (ILPs), are miniaturized medical devices which are entirely implanted into the patient's body, e.g. a heart's ventricle or atrium. Intracardiac pacemakers apply electrical stimulation in the form of pulses to the heart to generate a physiologically appropriate heartrate and/or in the form of shocks for cardioversion or defibrillation to restore a more normal heart rhythm. Additional functions of such implantable medical devices comprise providing other electrical or electromagnetic signals to the patient's body, e.g. the heart or its surrounding tissue, sensing electrical or electromagnetic signals or other physiological parameters of a pre-defined tissue, e.g. the heart and/or its surrounding tissue.

Leadless pacemakers largely aim to provide support as has been delivered for, e.g., bradycardia management in traditional, pocket based leaded implantable pulse generators (IPGs) through devices sized at ~10% of the total volume of legacy formats. While this miniaturization permits the placement of leadless implants within the blood volume of a patient's heart and provides reduced risk for infection through the elimination of leaded interfacing with the myocardium, an ILP has a small battery capacity due to the highly restricted device size. The requirements in terms of operational longevity are, however, the same. It is, therefore, paramount to minimize current draws from the battery during operation. That is to be achieved by ensuring optimal contact between the ILP electrode tip and the heart tissue. Additionally, the anchoring of the implantable device within the patient's tissue shall be permanently secure and any dislocation of the implantable device from the pre-defined target location shall be avoided.

Further, the above goals apply as well to medical devices/systems such as cardiac pacemakers or defibrillators of the conventional IPG type that comprise at least one implantable lead emanating from the medical generator device and terminating at a pre-defined target location (treatment location), in particular, to the implantable lead, since the implantable lead is fixed within the patient's tissue at the target location and provides electrical pulses to the pre-defined target location and/or transmits electrical signals detected at the target location to the generator device. In some use cases, the electrode of an implantable lead is deeply implanted within the myocardium of the right ventricle, e.g. within the septal wall, for stimulation of the left atrium. In such case, the electrode may penetrate the fascial tissue thereby reducing the retention force of this tissue so that an additional anchoring mechanism is desirable.

Document US 2019/0083779 A1 discloses an ILP which comprises a housing, an electrically insulative distal member, which is coupled directly to the housing and a tissue piercing electrode that extends helically from the housing. Alternatively, the ILP is coupled to the heart's tissue using fixation members, e.g. three tines, that extend from the distal end of the housing. The distal tips of the tines penetrate the heart tissue to a limited depth before elastically curving back proximally into the normally curved position. Such tines sometimes provide a fixation that is not well defined and do not support optimal contact between an ILP electrode tip and the tissue.

Alternatively, stiff screw-like or helical fixation members at the distal end of the lead are commonly used to anchor an implantable lead of a pacemaker in a patient's tissue. Such screws would have a high risk of dislocation if not only the screw but also the electrode tip was to penetrate the target tissue, thereby reducing the retention force of the fascial tissue to which an implantable device or electrode lead is often attached.

Accordingly, the objective is to provide secure anchor for an implantable lead or a leadless implant that utilizes the positive mechanical properties of intact fascial tissue and provides optimal contact of an electrode member to the pre-defined tissue at the target location.

The above objective is solved by an electrode assembly for an implantable lead or for a leadless implant having the features of claim 11 an implantable lead having the features of claim 9, a leadless implant having the feature of claim 11 as well as a fixation method of a medical device of the group comprising such implantable lead and such leadless implant having the features of claim 13.

In particular, the above objective is solved by an electrode assembly for an implantable medical lead or for a leadless medical implant, wherein the assembly comprises a tubular or cylindrical body extending in a longitudinal direction, wherein the assembly further comprises at least one spiral fixation member extending from the distal end of the body, wherein the spatial extension in radial direction of the at least one fixation member is configured such that, at zero-load, the respective fixation member protrudes beyond the greatest outer circumference of the body, wherein an electrode member is provided at the distal end of the body, wherein the electrode member is configured to receive electrical signals from and/or to transmit electrical signals to a tissue at a pre-defined target position within a patient's body, wherein one of the at least one fixation member forms the electrode member or the electrode member is an element formed separately from the at least on fixation member and is located at a distal end of the body.

The above assembly comprises a tubular or cylindrical body that extends in a longitudinal direction. The longitudinal direction is provided by the longitudinal axis of the tubular (hollow cylindrical) or cylindrical body. Dependent on the use of the assembly, the cylindrical body may form a distal head (portion) of an implantable lead or a body or distal body portion (housing or distal housing portion) of a leadless implant. Both possibilities are described below in detail.

The implantable lead may be used to transmit electrical signals from and/or to a tissue at a pre-defined target position within or at a patient's heart as a part of a pacemaker or defibrillator system, e.g., an implantable cardioverter-defibrillator (ICD). The generator device of the pacemaker or defibrillator system is electrically and mechanically connected to the lead, wherein the generator device may comprise a control unit, a generator unit for generating the electrical pulses and a power supply, e.g. a battery, and is configured to be implanted within the patient's body, as well, but with a distance to the distal end of the implantable lead.

The leadless implant may comprise a control unit, a generator unit for generating the electrical pulses and a power supply, e.g. a battery. The electrode member providing the electrical pulses to or receiving electrical signals from the tissue at the pre-defined target position directly projects from the body (housing) of the implant. Accordingly, the implant is configured to be implanted adjacent the pre-defined target position, for example within a ventricle or atrium of the patient's heart. The implant may be configured to provide electrical pacing and/or defibrillating signals to the patient's heart. Accordingly, the implant may be an implantable leadless pacemaker (ILP) or an implantable leadless cardioverter-defibrillator.

At the distal end, the body of the assembly comprises the electrode member. Generally, if the electrode member is separate from the fixation member, the electrode member may be a pin-shaped element extending through an electrically isolating section at the body and is electrically connected to a conductor and/or electrical components accommodated within the body. The electrode member projects from the distal end of the body. Alternatively, one fixation member forms the electrode member, wherein this fixation member is connected to an electrically conducting element extending through an electrically isolating section at the body. By the electrically conducting element, in this case, the functional unit “electrode/fixation member” is electrically connected to a conductor and/or electrical components accommodated within the body.

The present disclosure is directed toward overcoming one or more of the above-mentioned problems, though not necessarily limited to embodiments that do.

### SUMMARY

According to the invention, the electrode assembly further comprises at least one spiral fixation member extending from the distal end of the body, wherein, at zero-load, the respective fixation member protrudes in radial direction beyond the greatest outer circumference of the body. This means, for example, that the overall spatial extension in radial direction of all fixation members is greater than the maximum outer diameter of the body. The radial direction is perpendicular to the longitudinal direction. Each fixation member is formed as two-dimensional or three-dimensional spiral, e.g. its form is similar to at least a section of a mathematical spiral curve which emanates from a starting/reference point moving farther away as it revolves around the starting point in two or three dimensions, i.e. the distance of a point of the spiral curve is a function of the angle. The spiral may be an Archimedean (arithmetic) spiral. Further, the plane of the two-dimensional spiral may be perpendicular to the longitudinal direction and the three-dimensional spiral may evolves into the longitudinal direction. Since it may form a section of such mathematical curve, the fixation member spiral doesn't need to start from this starting point but can be thought of as continuing to this starting point. In embodiments, the electrode assembly may comprise one fixation member, two fixation members, three fixation members or more than three fixation members. If there are more than two fixation members, in one embodiment, they are about equally distributed along the circumference of the body's distal end of the electrode assembly.

With regard to the at least one fixation member it is realized, that the spatial extension in radial direction of the at least one fixation member is configured such that, at zero-load, the respective fixation member protrudes in radial direction beyond the greatest outer circumference of the body. This means that all fixation members protrude beyond this greatest outer circumference. For example, at the distal end of one fixation member (i.e. the end that is opposite to the attachment location of the fixation member to the body) has a distance d from the greatest outer circumference of the body in radial direction that is in the range of 0.5 mm to 5 mm, preferably 1 mm to 3 mm. This feature ensures permanently secure anchoring of the implantable lead or leadless implant. This is because the inner diameter of a catheter shaft (or a catheter comprising a catheter shaft) introducing the implantable lead or the leadless implant corresponds to the outer diameter of the greatest outer circumference of the body. Accordingly, as the at least one fixation member protrudes beyond this outer circumference, the at least one fixation member is compressed within the catheter shaft during insertion into the patient's body and advancing to the target location within the patient's body. During the final fixing step, the at least one fixation member is introduced into the tissue at the target location and the catheter shaft is retracted at the same time. At the beginning of this step, the at least one fixation member is still compressed by the partly retracted catheter shaft but already partly introduced into the tissue. The at least one fixation member expands in radial direction during further retraction of the catheter shaft and reaches its greatest radial dimension in its zero-load condition only after the catheter shaft is fully retracted from the at least one fixation member. However greater radial expansion leads to contraction in longitudinal direction thereby effecting strain in longitudinal direction that causes pulling of the assembly and thereby the electrode into the direction of the tissue. Thereby, the electrode may penetrate into the tissue or may at least be pressed with a higher contact pressure against the tissue surface. Accordingly, a permanent and reliable electrical contact of the electrode to the tissue is realized. Additionally, the expansion of the at least one fixation element further causes anchoring of the at least one fixation member behind/within a fascial tissue and/or trabecular meshwork covering the tissue (e.g. the myocardium), if fascial tissue or trabecular meshwork is present. Consequently, in this case the fascial tissue and/or the trabecular meshwork effect stable and secure fixation of the assembly and thereby of the implantable lead or the leadless implant.

The fixation member is attached to/fixed at/embedded within the assembly's body by being part of the tubular distal end of the body, by overmolding, by fixing with a cap that is pushed on from the distal end, by gluing and/or by connecting via a ring placed behind an undercut.

In one embodiment, the electrode assembly comprises one fixation member that is a three-dimensional spiral winding around a first (starting) point by an angle of at least 540° at zero-load, wherein the first point is located on the longitudinal axis of the body. This is a simple single fixation member that can be used for an implantable lead, for example. The at least one fixation member has the general shape of a three-dimensional spiral winding curve that starts at the first point which is located on the longitudinal axis of the body. As explained above, the fixation member doesn't need to start at this point by realizes a section of this curve. However, the end section covering an angle of at least 100°, or at least 150°, of the spiral protrudes beyond the greatest outer circumference of the body.

In one embodiment, the electrode assembly comprises at least two fixation members, wherein each fixation member is an arm winding two-dimensionally around a second (starting) point by at least 100°, for example by at least 150°, at zero-load, wherein the second point is located at the longitudinal axis of the body or in a central region at the distal end of the body. The central region is a central area at the distal end of the body (i.e. near or in the area of the longitudinal axis) that has a maximum diameter that is half the diameter of the greatest outer circumference of the body. This embodiment is particularly suitable for a leadless implant. In one embodiment, the pin-shaped electrode member is surrounded (encircled) by a collar-shaped element (i.e. a hollow cylindrical element) from which each arm projects (with a radial component) as a two-dimensional spiral.

In one embodiment, the electrode assembly comprises at least two fixation members, for example, at least three fixation members, wherein each fixation member is a wire winding three-dimensionally around a third (starting) point by at least 100° at zero-load, wherein the third point is located in an outer edge region at the distal end of the body. This embodiment is particularly suitable for a leadless implant, as well. In one embodiment, each fixation member projects from an area near the outer edge region at the distal end of the body. The outer edge region may be defined as a ring-shaped area starting at an inner diameter that is greater than half of the diameter of the greatest outer circumference of the body.

In one embodiment, the fixation member is made of a round wire or a flat wire. The fixation member consisting of a round wire may be produced easily and cost-effectively. The flat wire has the advantage that it may be cost-effectively made from sheet metal or tube e.g. via laser cutting.

As indicated above, in one embodiment, the at least two fixation members extend from a collar-shaped element (i.e. a hollow cylindrical element) located at the distal end of the body. For example, the collar-shaped element (hereinafter also referred to as “collar”) surrounds the pin-shaped electrode member extending therefrom. Due to its extension from the collar-shaped element the at least one fixation member has a pre-defined distance from the distal end of the body.

In one embodiment, the at least one fixation member comprises or consists of at least one material having a pre-defined minimum elastic modulus of 50 GPa, preferably at a temperature from 15 °C to 40 °C, and may be selected from the group comprising metals and corresponding alloys and plastics, for example, Nitinol, MP35N (Nickel-Cobalt-based alloy with significant additions of Chromium and Molybdenum), Tantalum, Titanium, Platinum-Iridium alloy and res. In one embodiment, the fixation member consists of a metal material that is covered by a plastic layer. Such material is biocompatible and provides the necessary elasticity of the fixation member for the above described deformation within the catheter shaft and recovering to its zero-load shape. The fixation member may be produced such that in the first step the material of the fixation member is cut from a round or flat semi-finished product and then drawn into its pre-defined shape (plastic deformation) that is adopted at zero-load.

In one embodiment, the fixation member comprises a hook at its first end portion, wherein the first end portion is opposite the second end portion, wherein the fixation member is attached to the body at its second end portion. The hook forms a section of the fixation member that is slightly angled (e.g. by 30° and more) with regard to the portion forming the second end and that has a length, for example, between 1 mm and 2 mm, in particular 1.5 mm. The hook may be, for example angled into the proximal direction. It provides a further improvement of a secure fixing of the implantable lead or the leadless implant to the tissue at the pre-defined target location as the hook digs into the patient's tissue, for example the fascial tissue or the trabecular meshwork. In one embodiment, each fixation member comprises a hook and in another embodiment every second fixation member comprises a hook.

The above objective is further solved by an implantable lead comprising the above described electrode assembly, wherein the electrode assembly is located at the distal end of the lead. As indicated above, the assembly may form a distal head of the implantable lead, for example a portion comprising an electrically insulating material insulating a central pin-shaped electrode member from another electrode member located with some distance in proximal direction. The body may form an electrically isolating portion for isolation of a central electrode member and may have a hollow cylindrical shape. The body may comprise further electrode members, for example ring-shaped electrode members at its shell surface. The implantable lead may further comprise an electrically insulating and covering sleeve extending almost along its full length (dimension in longitudinal direction) that is attached to the proximal end of the body. In one embodiment of the implantable lead, the lead comprises a connector at its proximal end, wherein the connector is electrically connected to the electrode member. The connector is used to electrically and mechanically connect the lead to the generator device providing and processing the signals (received and emitted signals, respectively).

The above objective is further solved by a leadless implant comprising the above described electrode assembly, wherein the electrode assembly is located at the distal end of the implant, wherein the assembly's body forms a distal end section of the implant's body (housing) or the full body (housing) of the implant. As indicated above, the implant comprises a control unit with a processor, a generator unit for generating the electrical pulses and a power supply, e.g. a battery. These elements/units are electrically connected and form an electronic circuit within the by implant body. The electrode member providing the electrical pulses to or receiving electrical signals from the tissue at the pre-defined target position directly projects from the body of the implant.

In one embodiment the leadless implant is an implantable leadless pacemaker or an implantable leadless defibrillator.

The above objective is further solved in a simple manner by a fixation method of a medical device of the group comprising an above described implantable lead according and an above described leadless implant to the patient's tissue at a pre-defined target location, comprising the following steps:
Providing the medical device within a catheter shaft or a catheter comprising a catheter shaft, wherein the at least one fixation member is covered by the distal end of the catheter shaft,
Introducing into and advancing the catheter shaft containing the medical device to the target location within the patient's body,
Introducing the fixation member into the tissue at the target location and simultaneously retracting the catheter shaft,
Removing the catheter shaft or the catheter comprising the catheter shaft from the patient's body.

The above objective is further solved in a simple manner by an implantation assembly for implanting a medical device of the group comprising an above described implantable lead and an above described leadless implant to the patient's tissue at a pre-defined target location, comprising a catheter shaft (or a catheter comprising a catheter shaft) configured to provide the medical device within the catheter shaft, wherein the at least one fixation member is covered by the distal end of the catheter shaft, wherein the catheter shaft containing the medical device is further configured to be introduced and advanced to the target location within the patient's body, to introduce the fixation member into the tissue at the target location and simultaneously retracting the catheter shaft, and to remove the catheter shaft (or the catheter comprising the catheter shaft) from the patient's body.

Additional features, aspects, objects, advantages, and possible applications of the present disclosure will become apparent from a study of the exemplary embodiments and examples described below, in combination with the Figures and the appended claims.

### BRIEF DESCRIPTION OF THE DRAWINGS

The present invention will now be described in further detail with reference to the accompanying schematic drawings, wherein

FIG. 1 shows a first embodiment of an electrode assembly for an implantable lead in a perspective side view at zero-load,

FIG. 2 depicts the embodiment of FIG. 1 during implantation located within a catheter shaft in a perspective side view,

FIGS. 3 to 4 two implantation steps of the embodiment of FIG. 1 in a perspective side view,

FIG. 5 a leadless implant when implanted in a patient's heart in a partial cross section,

FIG. 6 a first embodiment of a leadless implant in a side view at zero-load,

FIG. 7 the embodiment of FIG. 5 during implantation located within a catheter shaft in a side view,

FIG. 8 the embodiment of FIG. 5 in a top view at zero-load,

FIG. 9 a second embodiment of a leadless implant in a top view at zero-load,

FIG. 10 a third embodiment of a leadless implant in a side view at zero-load,

FIG. 11 a second embodiment of an electrode assembly in a perspective side view at zero load,

FIG. 12 the embodiment of FIG. 11 during implantation located within a catheter shaft in a perspective side view,

FIG. 13 a third embodiment of an electrode assembly during implantation located within a catheter shaft in a perspective side view,

FIG. 14 the embodiment of FIG. 13 in a perspective side view at zero load,

FIGS. 15 to 16 two implantation steps of the embodiment of FIG. 13 in a perspective side view,

FIG. 17 a fourth embodiment of an electrode assembly in a perspective side view at zero load,

FIG. 18 the embodiment of FIG. 17 during implantation located within a catheter shaft in a perspective side view,

FIG. 19 an extended embodiment of FIG. 7, and

FIG. 20 an alternative or extended embodiment of FIG. 1.

### DETAILED DESCRIPTION

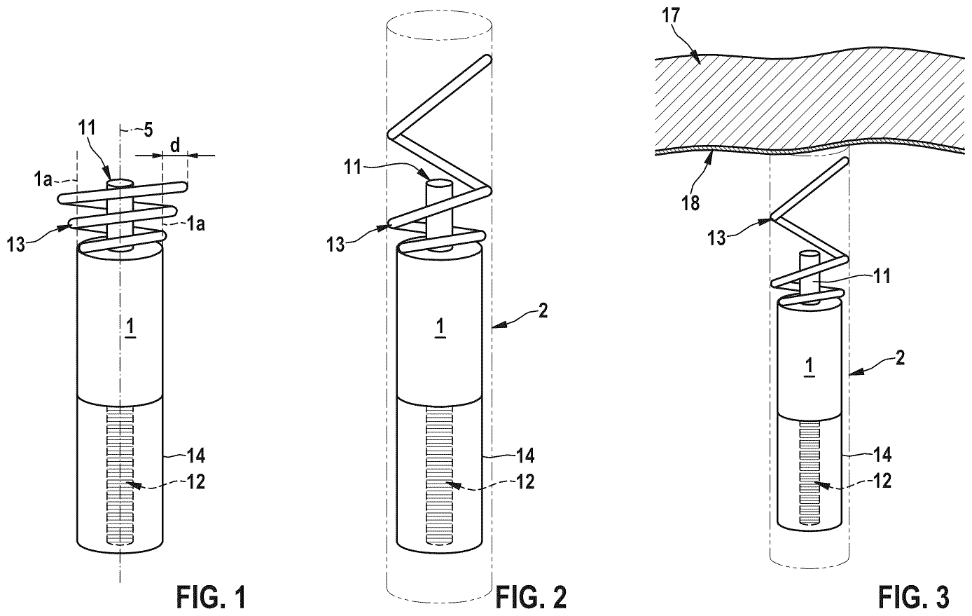

A first embodiment of an electrode assembly is shown in FIGS. 1 to 4. This electrode assembly is configured to form a distal end of an implantable lead that is partly shown in FIG. 1 to 4, as well. The electrode assembly comprises a body 1 having the form of a hollow cylinder with a longitudinal axis 5 (see FIG. 1) indicating the longitudinal direction. The body 1 is an isolating member for electrical isolation of an electrode member 11. The body 1 may comprise at least one further electrode member (e.g. a ring-shaped electrode member) at the surface but electrically isolated with respect to the electrode member 11. The pin-shaped electrode member 11 projects from the distal end of and extends through the body 1. The electrode member 11 is electrically and mechanically connected to a conductor 12 that connects the electrode member to a connector at a proximal end of the implantable lead. The conductor 12 is accommodated within and electrically isolated by an isolating sleeve 12 of the implantable lead located at the proximal end of the body 1. The electrode member 11 provides electrical pacing signals to the target tissue of the patient provided by a generator device that is connected to the implantable lead via the connector and/or receives electrical signals from the tissue at the target location and transmits them to the generator device.

The electrode assembly further comprises a fixation member 13 attached to the distal end of the body 1. The fixation member 13 is made of a Nitinol wire formed as a section of a three-dimensional spiral winding around a point at the longitudinal axis 5, wherein the fixation member 13 covers an angle of approximately 800° (more than two turns). At least a distal end portion of the fixation member 13 formed by approximately the last turn of the spiral fixation member 13 protrudes in radial direction beyond the greatest outer circumference of the body 1 marked in FIG. 1 by dashed lines 1a at zero-load. At the distal end section of the fixation member 13 it protrudes in radial direction by, for example, d=1.5 mm (see FIG. 1) beyond the greatest outer circumference.

For introduction of the implantable lead into the patient's body and advancing the lead to the target position where the lead is to be implanted, e.g. a location within the patient's heart, a catheter comprising a catheter shaft 2 is used, or an implantation assembly comprising the catheter shaft 2 (or comprising the catheter with the catheter shaft 2) is used. The implantable lead is accommodated within the catheter shaft 2 of such catheter as shown in FIG. 2. The inner diameter of the catheter shaft 2 corresponds approximately to the greatest outer circumference of the body 1 (see dashed lines 1a in FIG. 1). For clarity reasons the catheter shaft 2 is drawn little larger in FIGS. 2 and 3, in reality the inner surface of the catheter shaft 2 is accommodated directly adjacent the outer surface of the body 1.

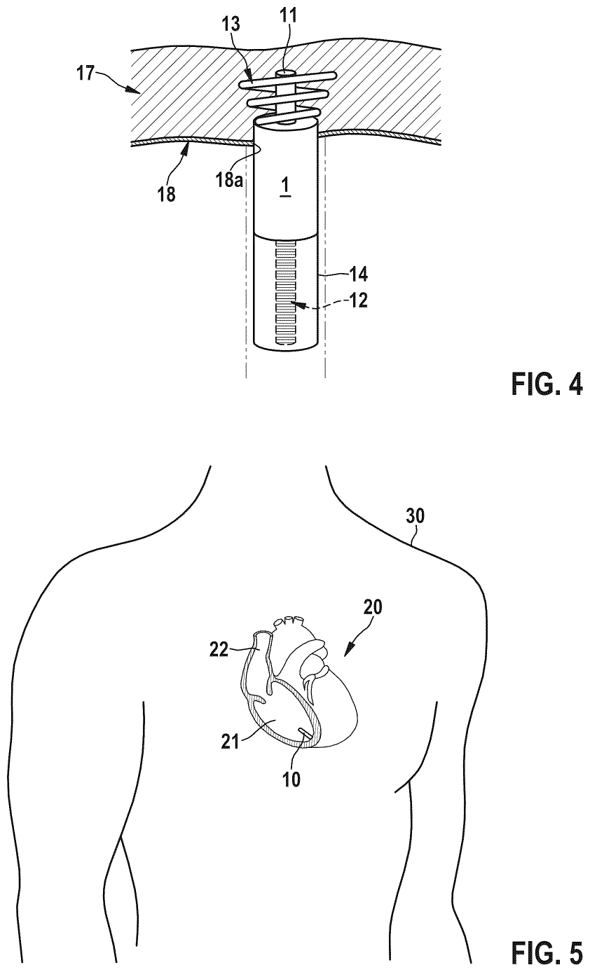

Within the catheter shaft 2 the fixation member 13 is compressed in radial direction so that it fits into the catheter shaft 2. As one can derive from FIG. 2, as a consequence, the fixation member 13 is deformed in longitudinal direction, as well such that the dimension (length) of the fixation member 13 in longitudinal direction is greater. When the system of implantable lead and catheter is has reached the target location during implantation (see FIG. 3), the implantable lead will be fixed at the respective tissue of the patient's heart comprising, for example, myocardium 17 and fascial tissue 18 covering the myocardium 17. The fixation member 13 and the electrode member 11 are pressed against the tissue and, if applicable, slightly rotated, such that they penetrate through the fascial tissue 18 into the myocardium 17. At the same time, the catheter is retracted (in proximal direction). Further, the fixation member 13 extends in radial direction as soon as the catheter shaft 2 does not limit its radial dimension any more. Thereby, the outer dimension in radial direction of the fixation member 13 gets greater than an opening 18a within the fascial tissue so that the fixation member 13 anchors behind the fascial tissue 18 at the rim of the opening 18a and is securely fixed after finishing the implantation (i.e. at zero-load). Additionally, due to the simultaneous reduction of the longitudinal dimension of the fixation member 13 when its outer radial dimension is extended, the electrode member 11 is “sucked” into the tissue (i.e. in longitudinal direction) thereby increasing the contact pressure and improving the electrode member's contact to the myocardium 17. The final position of the electrode member 11 and the fixation member 13 (at zero-load) within the myocardium where the assembly extends through the opening 18a within the fascial tissue 18 is shown in FIG. 4.

In the following, the invention is described with regard to a leadless ventricular pacemaker (ILP) as an embodiment of a leadless implant. FIG. 5 shows such ILP 10 as it is generally implanted within the heart 20 of a patient 30, wherein the heart 20 has a right ventricle 21 and a right atrium 22. ILP 10 may be configured to be implanted within the right ventricle 21 of the heart 20 and to stimulate using electrical signals provided by the electrode member this ventricle as well as may additionally sense intrinsic ventricular depolarizations and inhibit a ventricular pacing in response to a previously detected ventricular depolarization.

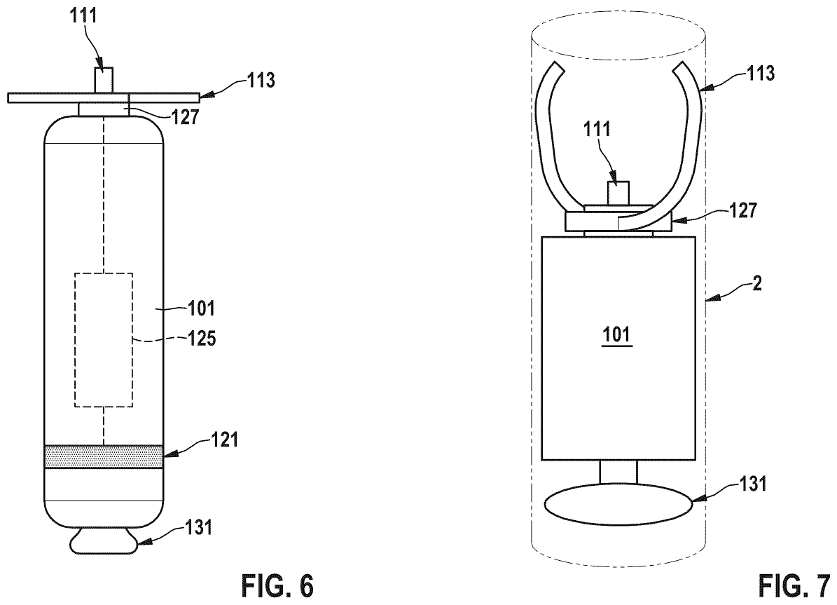

As shown in more detail in FIG. 6, a first embodiment of such ILP may include a body (housing) 101, two fixation members 113, and electrode members 111, 121 at a distal end of the body 101 and near a proximal end of the body 101. Electronic components 125 (see FIG. 6) are electrically connected to the electrode members 111, 121. The body 101 may have a pill-shaped cylindrical form factor in some examples. The fixation members 113 are configured to fix the ILP to heart's tissue. The fixation members 113 may be fabricated from a shape memory material, such as Nitinol. For example, as illustrated and described herein with respect to FIG. 5, fixation members 113 may be configured to anchor the ILP to heart 20 within the right ventricle 21. Although the ILP includes a plurality of fixation members 113 that are configured to stably engage the ILP to cardiac tissue in the right ventricle, it is contemplated that a pacemaker according to the present disclosure may be engaged with cardiac tissue in other chambers of a patient's heart 20 using this and below described types of fixation members. The body 101 further comprises a catheter engagement hitch 131 at the proximal end of the ILP.

The ILP may include two electrode members 111, 121, although more than two electrode members may be included on an ILP in other examples. As depicted in FIG. 6 electrode members 111, 121 may be spaced apart a sufficient distance to be able to detect various electrical signals generated by the heart 20, such as P-waves generated by atria and QRS complex generated by ventricles. The body 101 houses electronic components 125 including a battery, an electrical signal (pace signal) generator and a control unit having a processor. Electronic components 125 may include any discrete and/or integrated electronic circuit components that implement analog and/or digital circuits capable of producing the functions attributed to ILP described above.

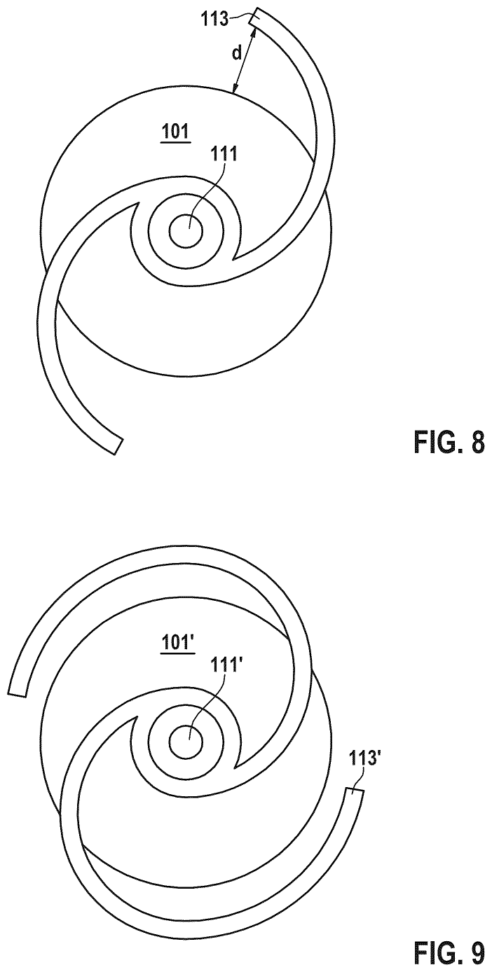

The two fixation members 113 are fixed to a shell surface of a hollow cylindrical collar 127 (or a collar-shaped element 127) projecting from the distal end of the body 101 and electrically isolating the pin-shaped electrode member 111 from the fixation members 113 and the body 101. As one can best derive from FIG. 8, each fixation member 113 has a two-dimensional spiral form with regard to one imaginary point located close to the longitudinal axis of the body 101 and covers an angle of about 150°. The attachment places of the two fixation members 113 are located on opposite sides of the shell surface of the collar 127 and the distal end of each fixation member 113 protrudes from the greatest outer circumference of the body 101 by a distance d in the range of 0.5 mm to 5 mm, preferably 1 mm to 3 mm (see FIG. 8). Accordingly, each fixation member 113 is deformed within the catheter shaft 2 as shown in FIG. 7 in radial direction (i.e. reduced in radial direction) and in longitudinal direction (i.e. extended in longitudinal direction) during implantation. This effects permanently secure fixation to the patient's heart tissue and an optimal contact of the electrode member 111 to the tissue similar to the embodiment shown with respect to FIG. 1 to 4.

The embodiment of an ILP shown in FIG. 9 is similar in structure and function to the embodiment shown in FIG. 6 to 8 with a body 101′ and a pin-shaped central electrode member 111′ but comprises two fixation members 113′ having a greater length than the fixation members 113 of the embodiment shown in FIG. 6 to 8. Accordingly, each fixation member 113′ covers an angle of approximately 220°.

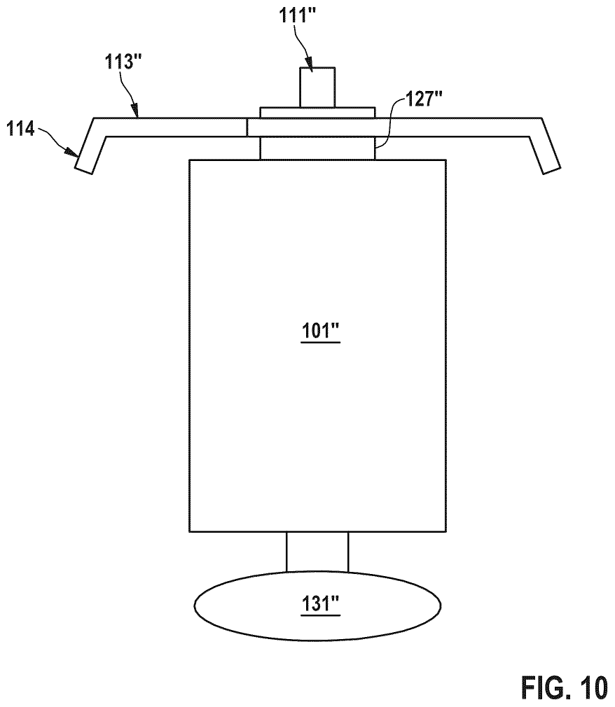

The embodiment of an ILP shown in FIG. 10 is similar in structure and function to the embodiment shown in FIG. 6 to 8 with a body 101″ and a pin-shaped central electrode member 111″ but comprises two fixation members 113″ having a hook 114 at the distal end of each fixation member 113″, wherein the hook-less portion of the fixation member 113″ is similar to the fixation member 113 of the embodiment shown in FIG. 6 to 8. The hook 114 forms an end portion that is angled with a minimum of 20° and a maximum of 45°, in particular with 30°. The hook is located at a first end portion with regard to a second end portion opposite the first portion, wherein the fixation member 113″ is attached to the collar 127″ at its second end portion. The hooks 114 further improve the secure fixing of the ILP to the target tissue as each one digs into the respective tissue.

The above embodiments of an electrode assembly (forming the distal part of the ILP) depicted in FIGS. 6 to 10 are generally also configured to be used with an implantable lead. The same applies to the electrode assembly shown in FIGS. 1 to 4 that may be used with an ILP in a similar manner.

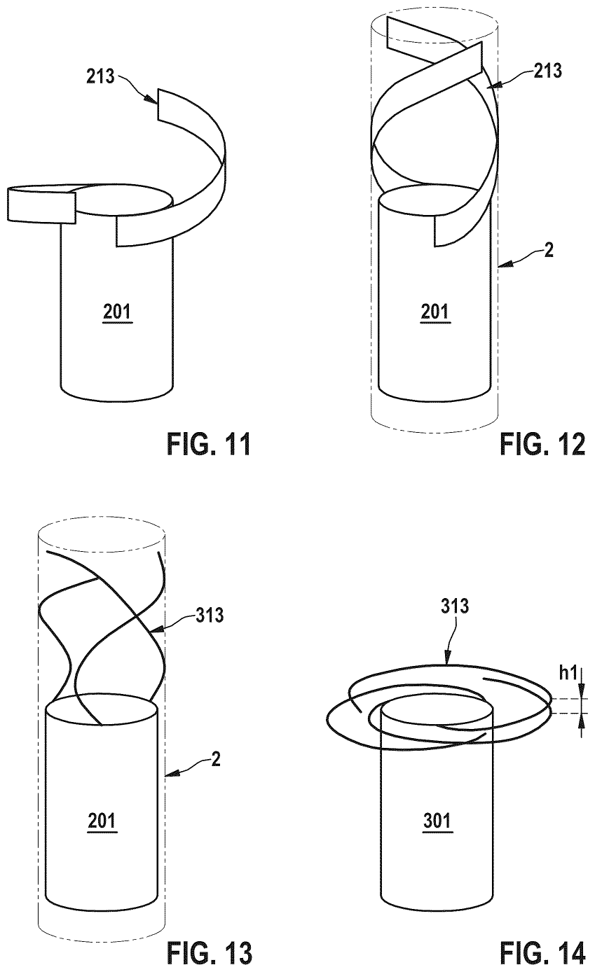

The following embodiments of electrode assemblies are drawn in FIGS. 11 to 18 in a very schematic way. Each embodiment shows the body 201, 301, 401 and omits—for clarity reasons —the separate electrode member which may be a central pin-shaped electrode member similar to the embodiment of FIGS. 1 to 4. It is contemplated with regard to all above and below discussed embodiments of electrode assembly, leadless implant or implantable lead that one of the at least one fixation member is configured to act as an electrode member. Accordingly, such fixation member consists of or comprises an electrically conducting material, for example MP35N, Tantalum, Titanium and/or Platinum-Iridium alloy, and is electrically connected to the conductor of the lead or the electronic components of the implant. The embodiments of electrode assemblies may be used with an implantable lead or a leadless implant, respectively.

The embodiment of an electrode assembly shown in FIGS. 11 and 12 comprises a cylindrical body 201 and two fixation members 213 formed by a flat wire. Each fixation member 213 is attached to a shell surface of the body 201 at its distal end and has the shape of a three-dimensional spiral winding section, wherein the reference (starting) point of the spiral winding is a point located close to the distal outer rim of the body 201. As one can derive from FIGS. 11 and 12, each fixation member protrudes in radial direction beyond the greatest outer circumference of the body (i.e. its shell surface) and is therefore compressed in radial direction when accommodated within the catheter shaft 2 during implantation (see FIG. 12).

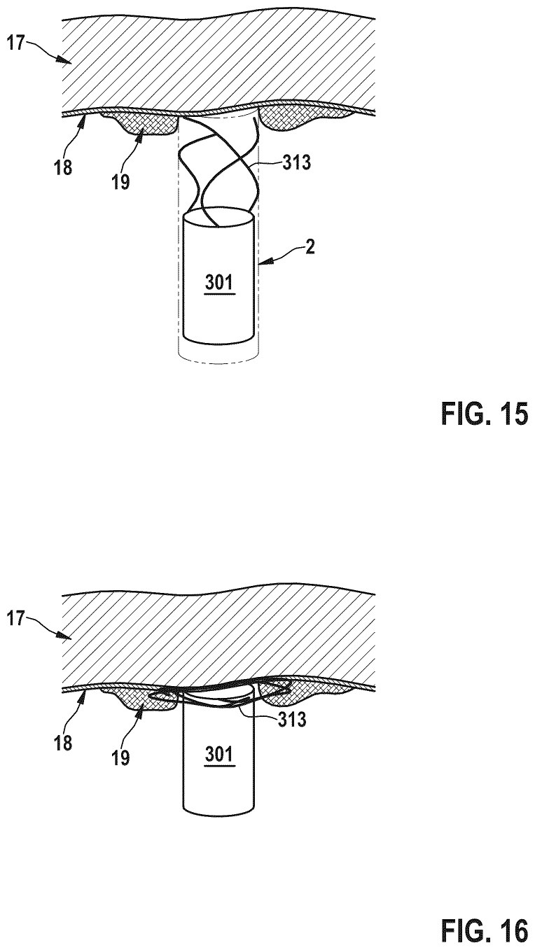

The embodiment of an electrode assembly shown in FIGS. 13 to 16 comprises a cylindrical body 301 and three fixation members 313 formed by a round wire. Each fixation member 313 is attached to a shell surface of the body 301 at its distal end and has the shape of a three-dimensional spiral winding section, wherein the reference (starting) point of the spiral winding is a point located close to the distal outer rim of the body 301. The attachment positions of the three fixation members 313 are equally distributed around the outer rim of the body 301 and have, therefore, a distance of 120°. As one can derive from FIGS. 13 and 16, each fixation member protrudes in radial direction beyond the greatest outer circumference of the body (i.e. its shell surface) and is therefore compressed in radial direction when accommodated within the catheter shaft 2 during implantation (see FIGS. 13 and 15).

FIGS. 15 and 16 show two time points during the fixing process of a medical device (leadless implant or implantable lead) having such electrode assembly at its distal end. FIG. 15 depict the situation in which the system of catheter with a catheter shaft 2 and medical device reaches the target location at a patient's heart comprising myocardium 17, fascial tissue 18 and trabecular meshwork 19. The fixation members 313 are introduced at the distal end of the catheter shaft 2 during simultaneous retraction of the catheter shaft 2 into the trabecular meshwork 19, fascial tissue 18 and myocardium 17 so that they anchor within and behind the trabecular meshwork 19 and the fascial tissue 18 as depicted in FIG. 16, wherein this Figure shows the state after finishing the fixing procedure at zero-load.

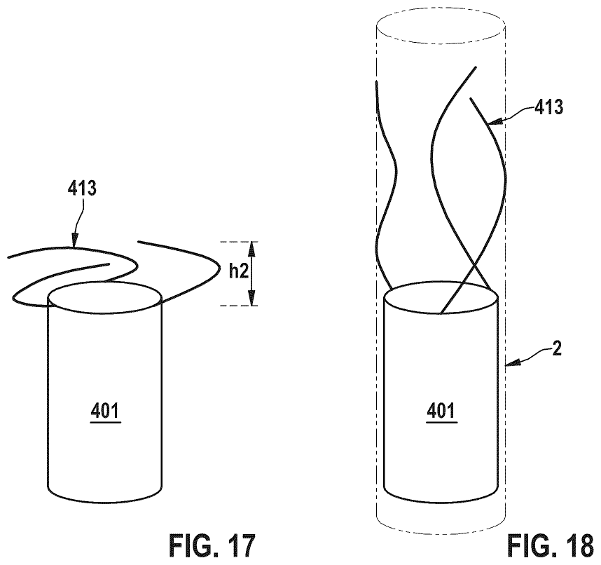

The embodiment shown in FIGS. 17 and 18 is similar to the embodiment depicted in FIGS. 13 to 16 except for the longitudinal extension of the fixation members with regard to the distal end face of the body at zero-load. The distal end face of the body may be defined as the distal area of the body with direct contact to the tissue. The electrode assembly of FIGS. 13 to 16 has fixation members 313 realizing a smaller extension h1 (see FIG. 14) with regard to the distal end face of the body 301 compared to the fixation members 413 with regard to the distal end face of the body 401 of the electrode assembly of FIG. 17 to 18 (see extension h2 in FIG. 17). For example, h 1 may be 0.5 mm, wherein h2 may be 2 mm. Accordingly, in a zero-load state after fixing the medical device to a patient's tissue, the fixation members 413 protrude farther into the tissue at the pre-defined target location (with respect to the longitudinal direction) than the fixation members 313.

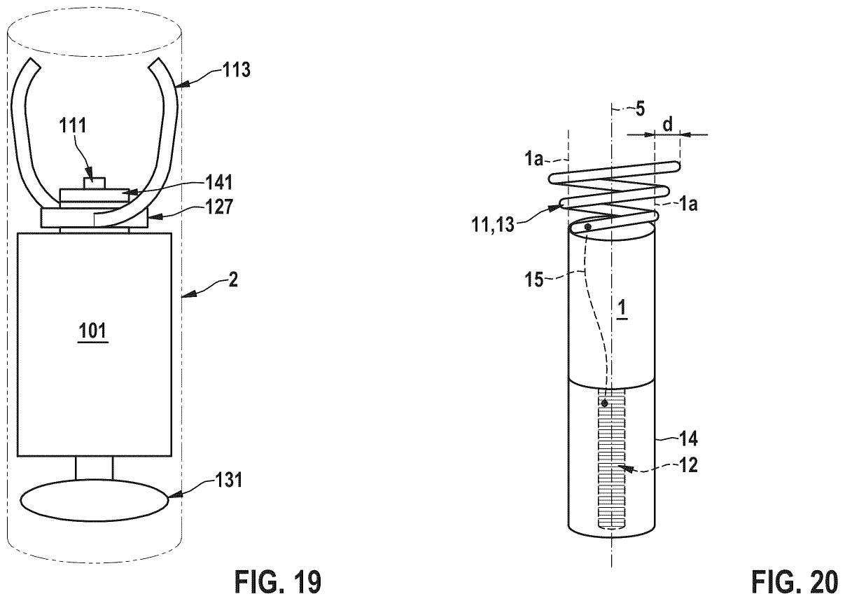

FIG. 19 shows an extended embodiment of FIG. 7, wherein the fixation member 113 is fixed to the assembly's body 101 with a cap 141 that is pushed on the distal end of the body 101.

FIG. 20 shows an alternative or extended embodiment of FIG. 1, wherein the fixation member 13 forms the electrode member 11, wherein this fixation member 13 is connected to an electrically conducting element 15 extending through an electrically isolating section (at/of the body) to the conductor 12.

All above embodiments of electrode assemblies and respective medical leadless implants and medical implantable leads can be permanently and securely fixed to the patient's tissue at the pre-defined target location and provide an optimal contact of the electrode member to the tissue.

It will be apparent to those skilled in the art that numerous modifications and variations of the described examples and embodiments are possible in light of the above teachings of the disclosure. The disclosed examples and embodiments are presented for purposes of illustration only. Other alternate embodiments may include some or all of the features disclosed herein. Therefore, it is the intent to cover all such modifications and alternate embodiments as may come within the true scope of this invention, which is to be given the full breadth thereof. Additionally, the disclosure of a range of values is a disclosure of every numerical value within that range, including the end points.

1. An electrode assembly for an implantable lead or for a leadless implant, wherein the assembly comprises a tubular or cylindrical body extending in a longitudinal direction, wherein the assembly further comprises at least one spiral fixation member extending from the distal end of the body, wherein the spatial extension in radial direction of the at least one fixation member configured such that, at zero-load, the respective fixation member protrudes in radial direction beyond the greatest outer circumference of the body, wherein an electrode member provided at the distal end of the body, wherein the electrode member is configured to receive electrical signals from and/or to transmit electrical signals to a tissue at a pre-defined target position within a patient's body, wherein one of the at least one fixation member forms the electrode member, or the electrode member is an element formed separately from the at least on fixation member and is located at a distal end of the body

2. The electrode assembly of claim 1, comprising one fixation member that is a three-dimensional spiral winding around a first point by an angle of at least 540° at zero-load, wherein the first point is located on the longitudinal axis of the body

3. The electrode assembly of claim 1, comprising at least two fixation members, wherein each fixation member is an arm winding two-dimensionally around a second point by at least 100° at zero-load, wherein the second point is located at the longitudinal axis of the body or in a central region at the distal end of the body.

4. The electrode assembly of claim 1, comprising at least two fixation members, wherein each fixation member is a wire winding three-dimensionally around a third point by at least 100°at zero-load, wherein the third point is located in an outer edge region at the distal end of the body.

5. The electrode assembly of claim 1, wherein the fixation member is made of a round wire or a flat wire.

6. The electrode assembly of claim 3, wherein the at least two fixation members extend from a collar-shaped element located at the distal end of the body.

7. The electrode assembly of claim 1, wherein the at least one fixation member comprises or consists of at least one material having a pre-defined minimum elastic modulus of 50 GPa and is selected from the group comprising metals and corresponding alloys and plastics.

8. The electrode assembly of claim 1, wherein the fixation member comprises a hook at its first end portion, wherein the first end portion is opposite the second end portion, wherein the fixation member is attached to the body at its second end portion.

9. An implantable lead comprising the electrode assembly of claim 1, wherein the electrode assembly is located at the distal end of the lead.

10. The implantable lead of claim 9, wherein the lead comprises a connector at its proximal end, wherein the connector is electrically connected to the electrode member.

11. A leadless implant comprising the electrode assembly of claim 1, wherein the electrode assembly is located at the distal end of the implant, wherein the assembly's body forms an end section of the implant's body.

12. The leadless implant of claim 11, wherein the leadless implant is an implantable leadless pacemaker or an implantable leadless cardioverter-defibrillator.

13. Fixation method of a medical device comprising an implantable lead and a leadless implant to the patient's tissue at a pre-defined target location, comprising the following steps:
Providing the medical device within a catheter shaft, wherein the at least one fixation member covered by the distal end of the catheter shaft,
Introducing into and advancing the catheter shaft containing the medical device to the target location within the patient's body,
Introducing the at least one fixation member the tissue at the target location and simultaneously retracting the catheter shaft,
Removing the catheter shaft from the patient's body.

14. Implantation assembly for implanting a medical device comprising an implantable lead and a leadless implant to the patient's tissue at a pre-defined target location, comprising:
a catheter shaft configured to provide the medical device within the catheter shaft-wherein the at least one fixation member-is covered by the distal end of the catheter shaft,
wherein the catheter shaft containing the medical device is further configured to be introduced and advanced to the target location within the patient's body, to introduce the at least one fixation member into the tissue at the target location and simultaneously retracting the catheter shaft, remove and to remove the catheter shaft from the patient's body.
